(19) United States
(12) Patent Application Publication
(10) Pub. No.: US 2012/0107835 A1
(43) Pub. Date: May 3, 2012

(54) METHOD FOR DETECTING BETA-AMYLOID USING AMINO ACID DIMERS

(75) Inventor: Hyo Bong Hong, Daejeon (KR)

(73) Assignee: ELECTRONICS AND TELECOMMUNICATIONS RESEARCH INSTITUTE, Daejeon (KR)

(21) Appl. No.: 13/268,347

(22) Filed: Oct. 7, 2011

(30) Foreign Application Priority Data
Nov. 3, 2010 (KR) 10-2010-0108824

(51) Int. Cl. G01N 33/82; C07C 237/12; C07D 495/04; C07D 209/20; C07C 229/36; G01N 33/566; C07C 323/59

(52) U.S. Cl. 435/7.5; 436/501; 435/7.92; 562/560; 562/556; 548/496; 562/448; 562/561; 548/304.1

(57) ABSTRACT

A method for detecting β-amyloid using an amino acid dimer instead of a secondary antibody binding to the β-amyloid primary antibody in an immunoassay based on an antigen-antibody reaction, and a kit for detecting β-amyloid including the dimer. The method is advantageous in that the dimer has good properties in stability, availability, and storage compared to antibodies and that it can detect various β-amyloids by using one kind of antibody, and thus may be effectively used in early diagnosis and research of Alzheimer's disease.

### CROSS-REFERENCE TO RELATED APPLICATION(S)

The present invention claims priority of Korean Patent Application No. 10-2010-0108824, filed on Nov. 3, 2010, which is incorporated herein by reference.

### FIELD OF THE INVENTION

The present invention relates to a method for detecting β-amyloid using an amino acid dimers, more particularly, to a method for detecting β-amyloid using an amino acid dimer instead of an antibody specifically binding to β-amyloid in an immunoassay based on an antigen-antibody reaction, and a kit for detecting β-amyloid comprising the amino acid dimer.

### BACKGROUND OF THE INVENTION

Alzheimer's disease (AD) is one of the aging-related neurodegenerative diseases and is characterized by memory loss. As the aging population grow, so does the number of AD patients; and AD has presented itself as a serious social challenge due to its social economical burdens.

As pathological characteristics of AD, senile plaques and neurofibrillary tangles in patient's brain have been observed, and owing to this, the loss of nerve cells has been clearly identified. Over 80% of a senile plaque consists of a toxic protein called β-amyloid (Aβ), which is currently considered as a major cause of AD. The Aβ protein consists of 40 or 42 amino acids cleaved from an amyloid precursor protein (APP) by β-secretase and γ-secretase. According to recent studies, Aβ consisting of 42 amino acids (also referred to Aβ 1-42 or Aβ42), rather than Aβ consisting of 40 amino acids (also referred to Aβ1-40 or Aβ 40), was founded to be a causative β-amyloid which is directly/indirectly associated with AD. Therefore, it needs to develop a method for detecting and measuring β-amyloid, particularly the causative Aβ, to diagnose AD precisely in its early stage and to treat it properly.

In general, immunoassays based on antigen-antibody reaction, e.g., ELISAs (Enzyme-Linked ImmunoSorbent Assays), western blots and the like, are used to detect Aβ. In spite of their advantages such as convenience for detection, the methods have problems in that a variety of different antibodies are required according to the kind of target Aβ, the synthesis of the antibodies used is complicated, and the resulting antibodies have low stabilities. Further, the antibodies may non-selectively bind to normal Aβ, other than causative Aβ, thereby generating analytical errors.

The inventors have endeavored to improve said immunoassays, and have achieved the present invention by confirming that β-amyloid can be measured by using an amino acid dimer instead of a conventional β-amyloid antibody, with distinguishing between Aβ40 and Aβ42.

### SUMMARY OF THE INVENTION

In view of the above, the present invention provides a novel method for detecting β-amyloid.

Further, the present invention provides a novel kit for detecting β-amyloid.

In accordance with a first aspect of the present invention, there is provided a method for detecting β-amyloid using an amino acid dimer instead of a secondary antibody binding to the β-amyloid primary antibody in an immunoassay based on an antigen-antibody reaction.

In accordance with a second aspect of the present invention, there is provided a kit for detecting β-amyloid including an amino acid dimer.

Previous immunoassays for detecting β-amyloid have used various antibodies according to the kind of β-amyloid, while the method of the present invention can detect β-amyloid by using one kind of antibody, e.g., amino acid dimer. In addition, the dimer is advantageous in stability, availability, and storage compared to antibodies, and can distinguish normal β-amyloid and causative β-amyloid, and thus may be effectively used in early diagnosis and research of AD.

### BRIEF DESCRIPTION OF THE DRAWINGS

FIG. 1 is a schematic diagram showing the analysis of β-amyloid in accordance with the present invention.

FIG. 2 presents a graph showing the analysis results of Aβ40 and Aβ42 in accordance with the present invention, wherein the horizontal axis displays 17 amino acid dimers used (see Table 1) and the vertical axis displays absorbance at 450 nm, and the rectangles (-▪-) in the graph represent the experimental results for β-amyloid 1-40, and the rhombus (-♦-), the experimental results for β-amyloid 1-42.

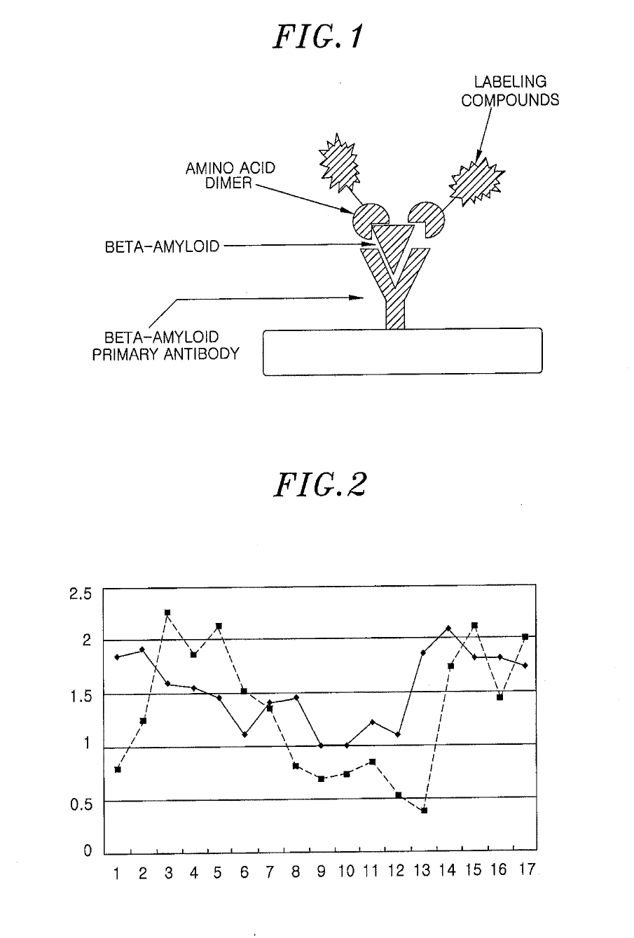

### DETAILED DESCRIPTION OF THE EMBODIMENTS

The present invention provides a method for detecting β-amyloid using an amino acid dimer instead of a secondary antibody binding to the β-amyloid primary antibody in an immunoassay based on an antigen-antibody reaction.

The immunoassay may be ELISA (Enzyme-Linked ImmunoSorbent Assay) or western blot.

As an example, a prior method for detecting β-amyloid using ELISA may be sandwich ELISA. The sandwich ELISA refers to a method comprising the steps of: coating a solid support or well with a β-amyloid primary antibody; applying an antigen (β-amyloid) thereto to allow antigen-antibody reaction; applying a secondary antibody linked with a label thereto to allow its binding to the resulting antigen-antibody complex; applying an enzyme linked with a material, which can bind with the label, to the resulting complex; and applying a substrate for the enzyme thereto to measure a change in color by absorbance.

The present invention is characterized by using an amino acid dimer instead of said secondary antibody binding to the β-amyloid primary antibody.

Examples of amino acid dimers, which can be used in the detection of β-amyloid in accordance with the present invention, are Ala-Arg, Arg-Ile, Arg-Leu, Arg-Met, Arg-Thr, Trp-Arg, Val-Arg, Cys-Ser, Thr-Cys, Tyr-Tyr and Val-Val. The amino acid dimers were selected considering the length of molecules, the harmony of hydrophobic and hydrophilic bonds, the presence of hydrogen bond, etc., and they bind to β-amyloid, especially Aβ40 and Aβ42. Of such amino acid dimers, Ala-Arg, Arg-Ile, Arg-Leu or Cys-Ser is preferred.

The amino acid dimer used in the method of the present invention may be conjugated with a label for immunoassay. The label may be any compound which is conventionally used in the art, preferably N-hydroxysuccinimide (NHS), carboimide (EDC), or biotin. N-hydroxysuccinimide may be conjugated with an amine group of the amino acid dimer, and EDC may be conjugated with a carboxylic acid group of the amino acid dimer. More preferably, biotin may be used.

Examples of biotin-conjugated amino acid dimers of the present invention include biotin-Ala-Arg, biotin-Arg-Ala, biotin-Arg-Ile, biotin-Ile-Arg, biotin-Arg-Leu, biotin-Leu-Arg, biotin-Arg-Met, biotin-Met-Arg, biotin-Arg-Thr, biotin-Thr-Arg, biotin-Trp-Arg, biotin-Val-Arg, biotin-Cys-Ser, biotin-Ser-Cys, biotin-Thr-Cys, biotin-Tyr-Tyr, and biotin-Val-Val.

The label (e.g., biotin) may bind to a certain enzyme-linked material which can bind with the label, and the addition of a substrate for the enzyme would result in a change in color. Thus, the presence of β-amyloid may be measured by absorbance. The enzyme, the material capable of binding to the label, and the substrate may be those well known in the art. Preferable examples of the enzyme include alkaline phosphatase (ALP) or peoxidase; preferable examples of the material capable of binding to the label include avidin, streptavidin or neutravidin; and preferable example of the substrate is TMB (tetramethylbenzidine). β-amyloid detectable by the method of the present invention is not limited to a certain kind, but preferable examples thereof include Aβ40 and Aβ42. The amino acid sequences of Aβ40 and Aβ42 are represented by SEQ ID NOs. 1 and 2, respectively, Aβ42 exhibits different biochemical properties from Aβ40, due to the differences in 41st and 42nd amino acids. In one embodiment, the method for detecting β-amyloid in accordance with the present invention includes the steps of: i) coating a solid support with a β-amyloid antibody and applying a test sample thereto to allow β-amyloid in the sample to form a β-amyloid-antibody complex; ii) applying a biotinylated amino-acid dimer to the β-amyloid-antibody complex obtained in i) to allow its binding to the complex; iii) applying an enzyme linked with avidin, streptavidin, or neutravidin to the complex obtained in ii); and iv) applying a substrate for said enzyme to the resulting reaction mixture to induce en enzyme-substrate reaction, stopping the reaction, and measuring absorbance of the reaction mixture.

In the step (i) of the method, a solid support or plate is coated with an antibody specific for β-amyloid to be tested (primary antibody), and a sample to be tested (“test sample”) is added thereto to allow β-amyloid in the sample to bind to the primary antibody, thereby immobilizing β-amyloid through the formation of β-amyloid-antibody complex. The β-amyloid may be Aβ340 or Aβ42, and the primary antibody is an antibody specific for Aβ40 or Aβ42, which may be prepared or obtained from commercial suppliers. In addition, the solid support or plate used in step (i) has no limitation in its type, but may preferably be a glass-type or a plate.

In step (ii), a biotin-conjugated amino acid dimer is added to the β-amyloid immobilized in step (i) to allow the binding of the dimer to the β-amyloid-antibody complex. In the biotin-conjugated amino acid dimer, the biotin moiety serves as a label, and the dimer moiety serves to replace a secondary antibody which was used in the conventional immunoassays, for example ELISA and western blot, by binding to β-amyloid.

In step (iii), an enzyme, which is linked to any one selected from the group consisting of avidin, streptavidin, and neuravidin, is added to the complex obtained in step (ii), thereby allowing the avidin, streptavidin, or neuravidin to bind with biotin in the complex. The avidin, streptavidin, or neuravidin plays a role in binding to biotin for detection, and the binding of avidin, streptavidin, or neuravidin with biotin is well known in the art. Examples of the enzymes available include alkaline phosphatase (ALP) or peoxidase, and preferably horseradish peroxidase (HRP) may be used.

In step (iv), a substrate for the enzyme is added to the resulting complex to induce an enzyme-substrate reaction, the reaction is stopped, and then, a change in color of the reaction mixture is measured by absorbance. The substrate as used herein may vary depending on the enzyme used in step (iii). For example, if alkaline phosphatase is used in step (iii), the substrate of step (iv) may be p-nitrophenyl phosphate (pNPP); and, if peroxidase, particularly horseradish peroxidase, is used in step (iii), the substrate of step (iv) may be tetramethylbenzidine (TMB), o-phenylenediamine dihydrochloride (OPD), or 2,2′-Azinobis[3-ethylbenzothiazoline-6-sulfonic acid]-diammonium salt (ABTS). In addition, the reagent for stopping the reaction may vary depending on the enzyme used in step (iii). For example, if alkaline phosphatase is used in step (iii), the reagent for stopping the reaction may be sodium hydroxide, and if peroxidase, particularly horseradish peroxidase, is used in step (iii), the reagent for stopping the reaction may be hydrochloric acid or sulfuric acid. In this step, when the substrate for the enzyme is added, the color of reaction mixture becomes changed to be expressed as a change in absorbance.

Furthermore, the present invention includes a kit for detecting β-amyloid comprising an amino acid dimer. As mentioned above, the amino acid dimer binds to a label, preferably biotin, and is used instead of a secondary antibody employed in a conventional immunoassay. Preferable examples of the amino acid dimer include Ala-Arg, Arg-Ile, Arg-Leu, Arg-Met, Arg-Thr, Trp-Arg, Val-Arg, Cys-Ser, Thr-Cys, Tyr-Tyr and Val-Val. The kit in accordance with the present invention may further includes any components widely used in the art, in addition to the amino acid dimer, e.g., a primary antibody for β-amyloid, a standard protein, an enzyme-linked material capable of binding to a label, a substrate for the enzyme, a washing solution, a reagent for stopping the reaction, etc.

Hereinafter, the present invention is described in more detail by way of Examples as follows. It is noted that the following Examples are given for the purpose of illustration only, and are not intended to limit the scope of the invention.

### Example 1

### Preparation of Biotin-Conjugated Amino Acid Dimer

### <1-1> Selection of Peptides

Peptides, which can be used instead of β-amyloid antibody in an immunoassay, were selected. Seventeen (17) amino acid dimers were chosen considering the length of molecule, the harmony of hydrophobic and hydrophilic bonds, the presence of hydrogen bonds, etc.

### <1-2> Construction of Biotin-Conjugated Amino Acid Dimers

Seventeen (17) biotinylated amino acid dimers were prepared by linking each amino acid dimer selected in Example <1-1> with biotin as a label by Peptrone, Inc. (Daejeon, Korea), and the composition of the biotinylated amino acid dimers is shown in Table 1.

TABLE 1

| | | Molecular |
|---|---|---|
| Type | Composition | weight |
| 1 | biotin-Ala-Arg | 471.23 |
| 2 | biotin-Arg-Ala | 471.23 |
| 3 | biotin-Arg-Ile | 513.27 |
| 4 | biotin-Ile-Arg | 513.27 |
| 5 | biotin-Arg-Leu | 513.27 |
| 6 | biotin-Leu-Arg | 513.27 |
| 7 | biotin-Arg-Met | 531.23 |
| 8 | biotin-Met-Arg | 531.23 |
| 9 | biotin-Arg-Thr | 501.24 |
| 10 | biotin-Thr-Arg | 501.24 |
| 11 | biotin-Trp-Arg | 586.27 |
| 12 | biotin-Val-Arg | 499.26 |
| 13 | biotin-Cys-Ser | 434.13 |
| 14 | biotin-Ser-Cys | 434.13 |
| 15 | biotin-Thr-Cys | 448.15 |
| 16 | biotin-Tyr-Tyr | 570.22 |
| 17 | biotin-Val-Val | 442.23 |

### Example 2

### Detection of β-Amyloid Using Amino Acid Dimers

Detection of β-amyloid was carried out using the biotinylated amino acid dimers prepared in Example 1, as follows.

Normal β-amyloid (Aβ40) and causative β-amyloid (Aβ42) were obtained from rPeptide (at Bogart, Ga., USA), and monoclonal antibodies specific for the β-amyloids(primary antibodies) were obtained from Covance (France). Each of the primary antibodies was mixed with sodium carbonate (pH 9.2) and the solution was added to a 96-well microplate for coating the wells (high-affinity F series, available from

NUNC, and then the microplate was washed with PBS or distilled water.

Meanwhile, each β-amyloid was dissolved in a sodium carbonate buffer (pH 9.4, Pierce) to a concentration of 1.0 mg/mL. For dilution, 100 μL of the β-amyloid solution was mixed with 9,900 μL of PBS. Each 200 μL of the resulting β-amyloid solution was dispensed into the wells of the 96-well microplate, and incubated for 4-hr at a room temperature so that β-amyloid binds to the primary antibody. Then, the plate was washed five times with PBS buffer (Pierce, at Rockford, Ill., USA), and 100 μL of the biotinylated amino acid dimer solution (amino acid dimer, dissolved in PBS to a concentration of 100 μM) was added to allow reaction for 1 hr. Each well was washed five times with PBS buffer, and 200 μL of streptavidin-HRP solution (diluted by 1:10,000, Pierce, at Rockford, Ill., USA) was added to allow reaction over 1 hr. After the reaction was completed, the plate was washed three times with PBS buffer, 100 μL of TMB substrate solution (KPL, USA) was added to allow reaction for 10-minute under a dark condition. Then, 50 μL of 15% hydrochloric acid was added to stop the reaction, and the absorbance of the reaction mixture at 450 nm was measured using a microplate reader (Bio-RAD, iMark).

The absorbance measurement results are shown in FIG. 2. As shown in FIG. 2, it was confirmed that both normal β-amyloid (Aβ40) and causative β-amyloid (Aβ42) can be measured using biotinylated amino acid dimers of the present invention, instead of secondary antibodies. In particular, in case of using dimers of Nos. 1, 3, 5 and 13, i.e., biotin-Ala-Arg, biotin-Arg-Ile, biotin-Arg-Leu and biotin-Cys-Ser, the difference of absorbance between Aβ40 and Aβ42 was very significant. These results indicate that the dimers of the present invention have different binding capacities to Aβ40 and Aβ42, and thus the dimers of the present invention are also useful in distinguishing Aβ40 and Aβ42.

While the invention has been shown and described with respect to the embodiments, it will be understood by those skilled in the art that various changes and modifications may be made without departing from the scope of the invention as defined in the following claims.

What is claimed is:

1. A method for detecting β-amyloid by an immunoassay based on an antigen-antibody reaction, which comprises using an amino acid dimer instead of a secondary antibody binding to the β-amyloid primary antibody.

2. The method of claim 1, wherein the immunoassay is ELISA (Enzyme-Linked ImmunoSorbent Assay) or western blot.

3. The method of claim 1, wherein the amino acid dimer is selected from the group consisting of Ala-Arg, Arg-Ile, Arg-Leu, Arg-Met, Arg-Thr, Trp-Arg, Val-Arg, Cys-Ser, Thr-Cys, Tyr-Tyr, and Val-Val.

4. The method of claim 3, wherein the amino acid dimer is selected from the group consisting of Ala-Arg, Arg-Ile, Arg-Leu, and Cys-Ser.

5. The method of claim 1, wherein the amino acid dimer is conjugated with a label.

6. The method of claim 5, wherein the label is biotin.

7. The method of claim 5, wherein the label-conjugated amino acid dimer is selected from the group consisting of biotin-Ala-Arg, biotin-Arg-Ala, biotin-Arg-Ile, biotin-Ile-Arg, biotin-Arg-Leu, biotin-Leu-Arg, biotin-Arg-Met, biotin-Met-Arg, biotin-Arg-Thr, biotin-Thr-Arg, biotin-Trp-Arg, biotin-Val-Arg, biotin-Cys-Ser, biotin-Ser-Cys, biotin-Thr-Cys, biotin-Tyr-Tyr, and biotin-Val-Val.

8. The method of claim 1, wherein the β-amyloid is β-amyloid 1-40 or β-amyloid 1-42.

9. The method of claim 1, comprising:
coating a solid support with a β-amyloid antibody and applying a test sample thereto to allow β-amyloid in the sample to immobilize a β-amyloid-antibody complex;
applying a biotinylated amino-acid dimer to the immobilized β-amyloid-antibody complex to allow its binding to the complex;
applying an enzyme linked with avidin, streptavidin, or neutravidin to the complex obtained in said applying the biotinylated amino-acid dimer to the immobilized β-amyloid-antibody complex; and
applying a substrate for said enzyme to the resulting reaction mixture to induce en enzyme-substrate reaction, stopping the reaction, and measuring absorbance of the reaction mixture.

10. The method of claim 9, wherein the β-amyloid is β-amyloid 1-40 or β-amyloid 1-42.

11. The method of claim 9, wherein the enzyme is alkaline phosphatase or peoxidase.

12. The method of claim 11, wherein the peroxidase is HRP (horseradish peroxidase).

13. The method of claim 9, wherein the substrate is TMB (tetramethylbenzidine).

14. A kit for detecting β-amyloid comprising an amino acid dimer.

15. The kit of claim 14, wherein the amino acid dimer is selected from the group consisting of Ala-Arg, Arg-Ile, Arg-Leu, Arg-Met, Arg-Thr, Trp-Arg, Val-Arg, Cys-Ser, Thr-Cys, Tyr-Tyr, and Val-Val.

16. The kit of claim 14, wherein the amino acid dimer is conjugated with a label.

17. The kit of claim 16, further comprising one or more components selected from the group consisting of a primary antibody for β-amyloid, a standard protein, an enzyme-linked material capable of binding to a label, a substrate for the enzyme, a washing solution, and a reagent for stopping the reaction.

18. The kit of claim 17, wherein the label is biotin.

19. The kit of claim 17, wherein the material capable of binding to a label is avidin, streptavidin, or neutravidin.

20. The kit of claim 17, wherein the enzyme is alkaline phosphatase or peoxidase.


SEQUENCE LISTING (2 sequences)

SEQ ID NO: 1 - protein, 40 aa, Homo sapiens
DAEFGHDSGFEVRHQKLVFFAEDVGSNKGAIIGLMVGGVV

SEQ ID NO: 2 - protein, 42 aa, Homo sapiens
DAEFGHDSGFEVRHQKLVFFAEDVGSNKGAIIGLMVGGVVIA